(19) United States
(12) Patent Application Publication
(10) Pub. No.: US 2005/0148991 A1
(43) Pub. Date: Jul. 7, 2005

(54) Device for using patient blood as diluent in administering pharmaceuticals

(76) Inventor: Douglas Giles Johnson, Arvada, CO (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/514,783

(22) PCT Filed: May 21, 2003
(86) PCT No.: PCT/US03/16279

(30) Foreign Application Priority Data
May 21, 2002 (US) 60382527

(51) Int. Cl. A61M 37/00; A61B 19/00; A61M 5/32

(52) U.S. Cl. 604/403; 604/406; 604/416; 604/6.15; 604/6.16

(57) ABSTRACT

The subject invention comprises an apparatus and a method for administering a pharmaceutical to a patient by collecting the patient's blood in a collection chamber and adding the drug thereto, followed by re-infusing the blood admix to the patient. The administration may be carried out on a batch or continuous basis.

### FIELD OF THE INVENTION

The present invention deals with devices and methods for administration of pharmaceuticals.

### BACKGROUND OF THE INVENTION

There are many situations where the administration of a drug in conventional diluents or formulations is impossible or made more difficult due to undesirable characteristics of the drug product. These could be due to, but not limited to, solubility issues of the drug, pain experienced by the patient during injection of the drug product, irritation of the venous tissues caused by either the drug or the characteristics of the drug product (e.g., pH, osmolality, etc.). Often, this problem is solved by administering the drug through a central line (e.g., peripherally inserted catheter or a port to the subclavian vein or superior vena cava). By injecting the drug product into a vessel with high blood flow, any negative effect of the drug product is quickly diluted to the point of insignificance. Even when central lines can address the difficulties they are expensive and their placement requires a surgical procedure. Therefore, a procedure and/or device that could minimize the impact of the above drug product issues without involving a central line would be desirable. Another way to deal with the issues of pain on infusion or solubility is by formulating the drug with solubilizing agents such as cyclodextrins or modified cyclodextrins or by manufacturing the drug product in a liposome or micelle or nanoparticle. None of these solutions is universal and each has increased manufacturing expenses. Some of the solubilizing excipients also have toxicity limitations. A procedure and/or device that could improve solubility without the added cost of manufacturing or risk of toxicity would be desirable.

Devices for the extracorporeal treatment of blood are known in the art, such as blood oxygenation devices, implantable biomedical devices, blood sampling or analysis or purification devices. Furthermore, extracorporeal treatments of blood have been described. Many such treatments are for the purpose of stabilizing the blood or blood component itself, for example, to prevent coagulation as with the addition of heparin (see e.g. Langer, et al. U.S. Pat. No. 4,981,596), to adjust hemoglobin levels, as in Shiino, et al., U.S. Pat. No. 4,981,596; and removing of fats or other substances from the blood prior to autotransfusion, as in Boehringer, et al., U.S. Pat. No. 5,133,703. Other examples include methods to oxygenate the blood; in the on-line mixing of a replacement fluid, e.g., saline, as a blood component such as red blood cells is being returned to a blood donor, as in Vishnoi, et al., International Patent Publication Number WO 01/17607; or for the exchange of ions in the blood with other ions as in Boehringer, et al., ibid., in the case of autotransfusion. In some instances the blood is treated in preparation for storage.

Devices and methods for storage and stabilization of drugs prior to delivery to a host are also known in the art, such as flexible multiple compartment drug containers, e.g. Smith, et al., U.S. Pat. No. 5,176,634; a multi-chamber container for storing a bicarbonate solution as in Duponchelle, et al., U.S. Pat. No. 6,475,529; and containers having selectively openable seals and/or peelable barrier means, e.g. Stone, et al., U.S. Pat. No. 4,519,499.

Erythrocyte-mediated delivery of drugs has been described, see Magnani, et al., Gene Therapy (2002) 9:749-51. The method requires the use of a specially designed apparatus and a multistep process for loading the drugs into the erythrocyte prior to administration.

Blood cardioplegia with RSR13 has been described in Kilgore, et al. Circulation (1999) 100[suppl II]:II-351-II-356. The disclosure of each patent and publication referred to herein, is incorporated by references herein it its entirety.

### SUMMARY OF THE INVENTION

The present invention provides an apparatus for administration of a pharmaceutical to a patient comprising a blood collection chamber, a means for adding a pharmaceutical to said blood collection chamber to form a blood admix, and a means for transporting the patient blood to the blood collection chamber and for transporting the blood admix to patient.

The transportation of patient blood to the blood collection chamber, the formation of the blood admix and the transportation of the blood admix to the patient can be carried out on a batch or continuous basis.

The means for adding the pharmaceutical to the blood collection chamber is a drug chamber in controllable fluid communication with the blood collection chamber, such as a tape that breaks along the length of the drug chamber, and the controllable fluid communication may comprise a frangible barrier that includes a sterilizing filter.

The means for transporting patient blood to and from the blood collection chamber may be blood tubing, which is connected to the patient's circulatory system. The tubing, in some embodiments, has a Y joint, wherein arms one and two of the tubing connect to the blood collection chamber, and arm three connects to the patient, wherein arm two that connects to the blood collection chamber optionally contains a filter by which the blood admix may be filtered prior to transport back to the patient.

In other embodiments, the Y joint contains a gating device, such as a stopcock, whereby blood admix flow to the patient is directed through either arm one or arm two with the filter.

The tubing may comprise a first tubing for transporting blood from the patient to the blood collection chamber, and a second tubing for transporting blood admix from the blood collection chamber to the patient. In some embodiments, the second tubing further comprises a filter. In other embodiments, the first tubing or second tubing comprises a gating means.

The present invention also provides a method for administering a pharmaceutical to a patient comprising transporting blood or a subfraction thereof from the patient to a blood collection chamber, adding a pharmaceutical to the blood collection chamber to form a blood admix, and returning the blood admix to the patient.

The method steps may be carried out on a batch or continuous basis. In other embodiments, the transportation of patient blood to the blood collection chamber is through a first tubing, and the transportation of blood admix to the patient is through a second tubing. In further embodiments, the patient blood or blood admix flow is controlled by gating devices on the first and second tubings.

In other embodiments, the transportation of patient blood blood or a subfraction thereof to the blood collection chamber and the transportation of blood admix to the patient is through the same tubing. The patient blood or blood admix flow may be controlled by a gating device on the tubing in some embodiments.

The pharmaceutical form used in the method may be selected from the group consisting of a solution, emulsion, suspension or solid.

In other embodiments, the pharmaceutical is added to the blood collection chamber via a port on the drug collection chamber. In other embodiments, the pharmaceutical is added to the blood collection chamber via a drug chamber in controllable fluid communication with the blood collection chamber.

In one embodiment, the pharmaceutical is 2-[4-[2-[(3,5-dimethylphenyl)amino]-2-oxoethyl]phenoxy]-2-methyl-propionic acid (RSR13) or a pharmaceutically acceptable salt.

In a further embodiment, the blood or a subfraction thereof is from the patient.

In yet another embodiment, the invention provides a method for reducing pain or irritation associated with the administration of a pharmaceutical in a blood vessel, comprising transporting blood or a subfraction thereof from the patient to a blood collection chamber, adding a pharmaceutical to the blood collection chamber to form a blood admix, and returning the blood admix to the patient, whereby pain or irritation associated with the administration of the pharmaceutical in a blood vessel is reduced.

In another embodiment, the invention provides a method for administering a pharmaceutical in a blood vessel in a manner that improves the mixability of the pharmaceutical in blood, comprising transporting blood or a subfraction thereof from the patient to a blood collection chamber, adding a pharmaceutical to the blood collection chamber to form a blood admix, wherein the mixability of the pharmaceutical is increased in blood compared to a the mixability of the pharmaceutical in a formulation for direct injection, and returning the blood admix to the patient, whereby the pharmaceutical is administered in a blood vessel.

### BRIEF DESCRIPTION OF THE DRAWINGS

FIG. 1 shows a depiction of an apparatus for administration of a pharmaceutical to a patient according to the present invention, which comprises a blood collection chamber and a drug port.

FIG. 2 shows a depiction of an apparatus for administration of a pharmaceutical to a patient according to the present invention, which comprises a blood collection chamber and a drug chamber.

### DETAILED DESCRIPTION OF THE INVENTION

The subject invention comprises a method and device which allows for the collection of an appropriate amount of blood in a container that is designed in such a way as to allow for the blood, once collected (and separated into components if desired), to be mixed with the drug of interest and then after mixing, re-infused into the patient. A particular fraction of the patient's blood may be separated by a membrane if, for example, only the plasma portion is needed to act as diluent. As used herein, blood refers to whole blood or any subfraction thereof, including, but not limited to red blood cells, white blood cells, plasma, and platelets. In a preferred embodiment, the blood is the patient's own blood.

In one embodiment, the subject invention provides a method for administration of a pharmaceutical, particularly administration in a blood vessel, wherein the solubility, miscibility, suspendibility or other mixibility of the pharmaceutical in blood is increased compared to a the mixability of the pharmaceutical in a formulation prepared for direct injection. This embodiment is particularly useful in situations where the pharmaceutical is not sufficiently soluble to permit its formulation for direct injection into a blood vessel, but is readily soluble in blood at therapeutic concentrations or is taken up by blood components (such as binding to albumin or crossing blood cell membranes). The increased mixability could be due to the inherent solubility in blood or complexing with components of the blood such as albumin or red blood cells.

In one embodiment, the subject invention provides a method for reducing the pain or irritation associated with the administration of a pharmaceutical, particularly administration in a blood vessel. In a further embodiment, the method provides substantially no pain, and/or substantially no irriation; i.e., substantially no cellular damage or substantially no necrosis. By mixing some or all of the drug with the patient's blood, the concentration of the free drug can be decreased to a level that is low enough that pain and/or irritation are reduced or even not observed.

Reduction in pain can be measured by any method known to those skilled in the art, including, but not limited to subjective self-report using a visual analogue scale, a quantified verbal descriptor scale, a comparison to prior injection experience, and an operator's global assessment of the subject's pain response.

As used herein, irritant refers to a therapeutic agent that may produce pain and/or inflammation at or near the administration site or along the path of the vein (phlebitis) by which it is administered. Examples of anti-cancer chemotherapeutic agents which are irritants include but are not limited to Carmustine, Dacarbazine, Etoposide, Plicamycin, Etoposice, Streptozocin and Tenoposide. Reduction in irritation can be measured by any method known to those skilled in the art, including, but not limited to reduction in cellular damage and reduction in necrosis.

In an early study in humans receiving 2-[4-[2-[(3,5-dimethylphenyl)amino]-2-oxoethyl]phenoxy]-2-methyl-propionic acid (RSR13) via injection in a peripheral vein, some recipients complained of pain near the site of injection and downstream. Therefore, the use of RSR13 and its physiologically acceptable salts in the present methods is contemplated. The preparation and uses for 2-[4-[2-[(3,5-dimethylphenyl)amino]-2-oxoethyl]phenoxy]-2-methyl-propionic acid and its physiologically acceptable salts has been described previously in U.S. Pat. Nos. 5,049,695; 5,122,539; 5,290,803; 5,432,191; 5,525,630; 5,648,375; 5,661,182; 5,677,330; 5,705,521; 5,872,888; and 5,927,283, and pending U.S. patent application Ser. No. 10/082,130, filed Feb. 25, 2002.

Other pharmaceuticals contemplated for use in the present method are vesicants. By vesicant is meant a chemotherapeutic agent which is topically toxic. If inadvertently delivered outside of a vein, a vesicant has the potential to cause pain, cellular damage including cellulitis, tissue destruction (necrosis) with formation of a sore or ulcer and sloughing of tissues that may be extensive and require skin grafting. Examples of anti-cancer chemotherapeutic agents that are vesicants include but are not limited to RSR13, Amsacrine, Dactinomycin, Daunorubicin, Doxorubicin, Idarubicin, Mechlorethamine, Mitomycin C, Vinblastine, Vincristine and Vindesine.

The drugs administered in the present methods may include formulation, carriers, buffers, diluents, and other pharmaceutically acceptable excipients such as mannitol, sorbitol, lactose, sucrose and the like. With reference to RSR13, suitable formulations are described in copending U.S. patent application Ser. No. 10/120,848, incorporated by reference herein in its entirety.

In another embodiment, the subject invention comprises an apparatus for administration of a pharmaceutical to a patient comprising a blood collection chamber, means for adding a pharmaceutical to the blood collection chamber to form a blood admix, and means for transporting the patient blood to the blood collection chamber and for transporting the blood admix to patient. The method of transporting patient blood to the collection chamber, adding the pharmaceutical and transporting the blood admix back to the patient can be carried out on a batch or continuous basis.

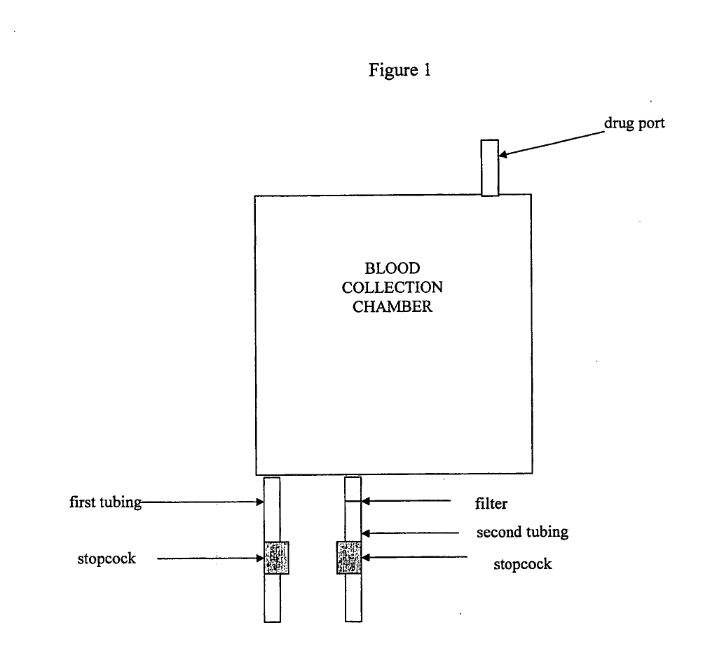

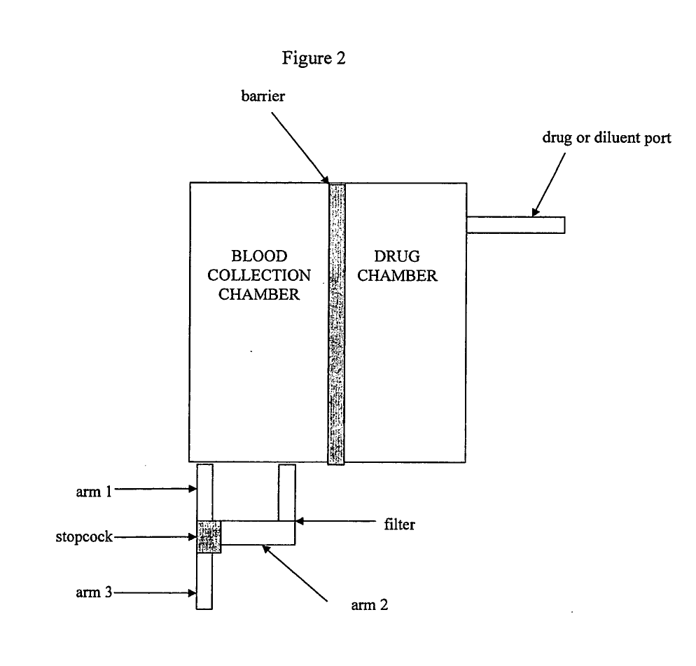

The blood collection chamber can be made of any material that is compatible with mammalian (e.g., human) blood. In a preferred embodiment, the chambers of the apparatus are flexible bags. If needed, an appropriate amount of a blood stabilizing agent (e.g. an anti-clotting agent) can be present in the blood collection chamber or added during the collection process. The pharmaceutical can be added to the blood collection chamber via an injection port into the blood collection chamber (FIG. 1), or via the opening or breaking of a barrier between the blood collection chamber and a drug chamber, whereby the drug chamber is placed in fluid communication with the blood collection chamber (FIG. 2). The drug in the drug chamber could be present as a solution, as an emulsion, as a suspension or as a solid. There may be a port on the drug chamber for adding drug to the drug chamber. If, for stability reasons, the drug is best present as a solid and if there is a need to dissolve the drug prior to the mixing with the blood, then a diluent can be added through the port (FIG. 2). Additionally, if the drug must be stored separately from the blood collection chamber, the drug chamber design permits such separate storage of the drug prior to being sent to the patient for use.

The frangible barrier between the blood collection chamber and the drug chamber may be designed so that when the breakage occurs, the drug is channeled through a sterilizing filter to prevent the contamination of the blood admix. The frangible barrier may also comprise a tape that breaks along the length of the drug chamber, permitting the mixing of blood and pharmaceutical. Such a peelable barrier is described in U.S. Pat. No. 4,519,499. Additional chambers are contemplated in the device, for example, a diluent chamber. A frangible barrier between the drug chamber and the diluent chamber may allow for preparation of a drug solution prior to the admixing of the drug with the blood.

The means for transporting patient blood to and from the blood collection chamber can be blood tubing. In one embodiment, the tubing has a Y joint (FIG. 2), and arms one and two of the tubing separately connect to the blood collection chamber, and also connect to each other and the third arm that connects to the patient at the central joint of the Y. Arms one or two optionally contain a filter by which the blood admix may be filtered prior to transport back to the patient. The filter pore size would be such that blood components would not be retained but any unusually large particle would be filtered out. The central joint typically contains a gating device, whereby blood flow to the patient is directed through either arm one or arm two. The gating device may be, for example, a stopcock.

In another embodiment (FIG. 1), the transportation of blood and blood admix is accomplished by a first tubing for transporting blood from the patient to the blood collection chamber, and a second tubing for transporting blood admix from the blood collection chamber to the patient. The first or second tubing may contain a filter and a gating means, such as a stopcock.

The subject invention also comprises a method for administering a pharmaceutical to a patient comprising transporting blood from the patient to a blood collection chamber, adding a pharmaceutical to the blood collection chamber to form a blood admix, and returning the blood admix to the patient. The method may be carried out on a batch or continuous basis.

1. An apparatus for administration of a pharmaceutical to a patient comprising:
a) a blood collection chamber;
b) means for adding a pharmaceutical to said blood collection chamber to form a blood admix; and
c) means for transporting the patient blood to the blood collection chamber and for transporting the blood admix to patient.

2. The apparatus of claim 1, wherein the transportation of patient blood to the blood collection chamber, the formation of the blood admix and the transportation of the blood admix to the patient is carried out on a batch basis.

3. The apparatus of claim 1, wherein the transportation of patient blood to the blood collection chamber, the formation of the blood admix and the transportation of the blood admix to the patient is carried out on a continuous basis.

4. The apparatus of claim 1, wherein said means for adding the pharmaceutical to the blood collection chamber is a drug chamber in controllable fluid communication with the blood collection chamber.

5. The apparatus of claim 4, wherein the controllable fluid communication comprises a frangible barrier that includes a sterilizing filter.

6. The apparatus of claim 4, wherein the controllable fluid communication comprises a tape that breaks along the length of the drug chamber.

7. The apparatus of claim 1, wherein the means for transporting patient blood to and from the blood collection chamber is blood tubing, which is connected to the patient's circulatory system.

8. The apparatus of claim 7, wherein the tubing has a Y joint, wherein arms one and two of the tubing connect to the blood collection chamber, and arm three connects to the patient, wherein arm two that connects to the blood collection chamber optionally contains a filter by which the blood admix may be filtered prior to transport back to the patient.

9. The apparatus of claim 8, wherein the Y joint contains a gating device, whereby blood admix flow to the patient is directed through either arm one or arm two with the filter.

10. The apparatus of claim 9, wherein the gating device is a stopcock.

11. The apparatus of claim 7, wherein the tubing comprises a first tubing for transporting blood from the patient to the blood collection chamber, and a second tubing for transporting blood admix from the blood collection chamber to the patient.

12. The apparatus of claim 11, wherein the second tubing further comprises a filter.

13. The apparatus of claim 11, wherein the first tubing or second tubing comprises a gating means.

14. A method for administering a pharmaceutical to a patient comprising:
a) transporting blood or a subfraction thereof from the patient to a blood collection chamber;
b) adding a pharmaceutical to the blood collection chamber to form a blood admix; and
c) returning the blood admix to the patient.

15. The method of claim 14, wherein the steps are carried out on a batch basis.

16. The method of claim 14, wherein the steps are carried out on a continuous basis.

17. The method of claim 14, wherein the transportation of patient blood to the blood collection chamber is through a first tubing, and the transportation of blood admix to the patient is through a second tubing.

18. The method of claim 17 wherein the patient blood or blood admix flow is controlled by gating devices on the first and second tubings.

19. The method of claim 15, wherein the transportation of patient blood blood or a subfraction thereof to the blood collection chamber and the transportation of blood admix to the patient is through the same tubing.

20. The method of claim 19, wherein the patient blood or blood admix flow is controlled by a gating device on the tubing.

21. The method of claim 14, wherein the pharmaceutical form is selected from the group consisting of a solution, emulsion, suspension or solid.

22. The method of claim 14, wherein the pharmaceutical is added to the blood collection chamber via a port on the drug collection chamber.

23. The method of claim 14, wherein the pharmaceutical is added to the blood collection chamber via a drug chamber in controllable fluid communication with the blood collection chamber.

24. The method of claim 14, wherein the pharmaceutical is 2-[4-[2-[(3,5-dimethylphenyl)amino]-2-oxoethyl]phenoxy]-2-methyl-propionic acid (RSR13) or a pharmaceutically acceptable salt.

25. The method of claim 14, wherein the blood or a subfraction thereof is from the patient.

26. A method for reducing pain or irritation associated with the administration of a pharmaceutical in a blood vessel, comprising:
a) transporting blood or a subfraction thereof from the patient to a blood collection chamber;
b) adding a pharmaceutical to the blood collection chamber to form a blood admix; and
c) returning the blood admix to the patient,
whereby pain or irritation associated with the administration of the pharmaceutical in a blood vessel is reduced.

27. A method for administering a pharmaceutical in a blood vessel in a manner that improves the mixability of the pharmaceutical in blood, comprising:
a) transporting blood or a subfraction thereof from the patient to a blood collection chamber;
b) adding a pharmaceutical to the blood collection chamber to form a blood admix, wherein the mixability of the pharmaceutical is increased in blood compared to a the mixability of the pharmaceutical in a formulation for direct injection; and
c) returning the blood admix to the patient,
whereby the pharmaceutical is administered in a blood vessel.
